(19) United States
(12) Patent Application Publication
(10) Pub. No.: US 2014/0038997 A1
(43) Pub. Date: Feb. 6, 2014

(54) PHARMACEUTICAL COMPOSITION OF VINFLUNINE WHICH IS INTENDED FOR PARENTERAL ADMINISTRATION PREPARATION METHOD THEREOF AND USE OF SAME

(71) Applicant: PIERRE FABRE MEDICAMENT, Boulogne-Billancourt (FR)

(72) Inventors: Elie LEVERD, Castres (FR); Joel BOUGARET, Francarville (FR); Marie-Dominique IBARRA, Saubens (FR)

(73) Assignee: PIERRE FABRE MEDICAMENT, Boulogne-Billancourt (FR)

(21) Appl. No.: 14/048,985

(22) Filed: Oct. 8, 2013

Related U.S. Application Data
(62) Division of application No. 12/846,334, filed on Jul. 29, 2010.
Application No. 12/846,334 is a continuation-in-part of application No. 10/584,445, filed on Jun. 22, 2006, now abandoned.

(51) Int. Cl. A61K 9/00; A61K 31/475

(52) U.S. Cl. CPC A61K 9/0019; A61K 31/475
USPC 514/281

(57) ABSTRACT

The invention relates to a pharmaceutical composition of vinflunine in the form of a stable sterile aqueous solution of a water-soluble salt of vinflunine with a pH of between 3 and 4. The invention also relates to the method of preparing said composition and to the use thereof as a parenterally-administered medicament for the treatment of cancer.

The present invention relates to a pharmaceutical composition for the parenteral administration of vinflunine.

Study of the antineoplastic properties of the alkaloids from Vinca rosea (Apocynacea family) has already made it possible to demonstrate the advantageous activities of compounds of indole structure, for instance vincristine, vinblastine or derivatives thereof, for instance vinflunine: 20′,20′-difluoro-3′,4′-dihydrovinorelbine of formula (a) below:

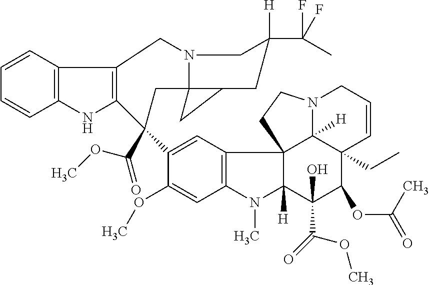

described in patent EP 0 710 240.

However, the development of injectable formulations of these active principles has always come up against problems associated with their stability in aqueous solution.

For many years, only the lyophilized form was marketed. Since it required an extemporaneous reconstitution with the contents of a solvent phial before administration, the lyophilisate presented major drawbacks associated with the hazards arising from handling it:
risk of reconstitution being performed incorrectly, during which fine droplets of product are generated, which may contaminate the person(s) performing the treatment, or the premises,
use of a poor amount of solvent or of an inappropriate amount of active principle if the pharmaceutical specialty is presented in different bottles corresponding to different unit doses.

This latter point is particularly important. It illustrates the potential possibilities of a non-therapeutic dose being administered to the patient or of exposure of the patient to an accidental overdose.

U.S. Pat. No. 4,619,935 suggested the possibility of formulating ready-to-use injectable solutions for Vinca alkaloids.

However the formulations used are complex. They comprise, in addition to the active principle:
a sugar or a sugar-based polyol, for instance mannitol,
an acetate buffer, to maintain the pH of the solution in the range 3.0-5.0 and more particularly in the range 4.4-4.8. Its molarity is between 0.02 and 0.0005 M and preferably between 0.01 and 0.002 M,
antimicrobial preserving agents.

It should be noted that, despite the stabilizing effect attributed to the acetate buffer, which makes it possible to prevent any degradation due to a change in pH caused by the decomposition of the alkaloids, the formulation that was the subject of the invention had a stability of only one year at 5° C.

The complexity of the patented formulations is increasing: patent FR 2 653 998 describes a pharmaceutical composition for parenteral use, containing an alkaloid of bis-indole type such as vincristine, vinblastine or 5′-nor-anhydrovinblastine. It is characterized in that it comprises, in aqueous solution, a zinc complex of an alkaloid salt of bis-indole type, a divalent metal gluconate and a preserving agent dissolved in a monohydric or polyhydric alcohol.

The stability indicated for these compositions is at least 24 months when they are stored in a refrigerator.

European patent EP 0 298 192 presents the favourable effect of ethylenediaminetetraacetic acid salts, in particular the sodium salt, on the stability of aqueous solutions of dimeric Vinca alkaloids. These aqueous solutions are buffered with an acetate buffer in order to maintain the pH between 3.0 and 5.5 and preferably between 4.0 and 5.0.

Under these conditions, with regard to the specifications adopted (alkaloid content of between 90% and 110% of the theoretical content), the solution remains stable for 30 months at a temperature of 2 to 8° C.

Canadian patent 2 001 643, relating to an injectable solution of vincristine, also emphasises the need to use an acetic acid/sodium acetate buffer to maintain the pH of the solution between 3.5 and 5.5, and more particularly between 4.0 and 4.5. The formulation described in the invention is stable for 18 months at 5° C., and may even be stable for 24 months at 5° C.

There is no example in CA 2 001 643 that a formulation without buffer and polyols and/or sugar is indeed providing the disclosed properties. The only examplified formulation of vincristine contains both a buffer and mannitol.

The specification indicating that vincristine solution may contain minor amounts of sugars and agents to buffer the pH means only that said excipients are not incompatible with the formulation. Indeed the wording that the formulation “consist essentially of” leaves to open door to other excipients rather than providing an incentive to avoid adding other excipients. The amount of degradation products in the tested samples of the solution of Example I table 5 which contain mannitol is raising e.g. from 2.4% to 3.5%, which constitutes an increase of 25 to 46% of the amount of impurities over days. Therefore, this solution is not very stable while containing mannitol. The product also loses activity by 3% over 7 days only. Therefore, the one skilled in the art would have thought that removing mannitol would have a strong impact on the stability of the solution; he would not have considered the option of removing mannitol and/or the pH buffer. This is confirmed by the fact that Vincristine sulphate parenteral formulation which is commercialized under the tradename ONCOVIN® contains mannitol and buffer according to the Label (dated July 1999).

Vinflunine ditartrate, or 20′,20′-difluoro-3′,4′-dihyrovinorelbine L(+)-tartrate, is a white powder that must be stored at a negative temperature, below −15° C., under an atmosphere of an inert gas such as nitrogen or argon.

It has been found, entirely unexpectedly, that vinflunine ditartrate is much more stable once it is dissolved in water than in pulverulent form.

Specifically, the injectable aqueous solution is stored at a positive temperature, of between +2° C. and +8° C. This is entirely surprising since it is well known that chemical degradation reactions take place more easily in liquid medium than in the solid state.

The present invention thus relates to a vinflunine pharmaceutical composition, characterized in that it is in the form of a stable and sterile aqueous solution of a water-soluble vinflunine salt at a pH of between 3 and 4.

The subject of the invention is based on the extraordinary simplicity of the formulation, which contrasts with the compositions described in the patents initially recalled.

Advantageously, the vinflunine salt is vinflunine ditartrate.

Advantageously, the pharmaceutical composition according to the present invention is in the form of a stable, sterile and apyrogenic, ready-to-use, injectable aqueous solution.

Advantageously, the composition according to the present invention does not contain any preservatives, or sugar or sugar alcohol such as mannitol.

In a first embodiment of the present invention, the pharmaceutical composition according to the present invention is in the form of a simple aqueous solution of vinflunine ditartrate, without addition of buffer solution. The composition thus consists of vinflunine ditartrate and water for injection.

As known by the one skilled in the art, water for injection is very pure apyrogenic water, obtained by distillation of water.

Advantageously, the pH of this solution is equal to 3.5.

In a second embodiment of the present invention, the pharmaceutical composition according to the present invention comprises a pH buffer system in order to maintain the pH between 3 and 4. Even more advantageously, the pharmaceutical composition according to the present invention consists of vinflunine ditartrate, water for injection and a pH buffer in order to maintain the pH between 3 and 4. Advantageously, the molarity of the pH buffer system is between 0.002 M and 0.2 M.

Advantageously, the buffer system consists of an acetic acid/sodium acetate buffer or a citric acid/sodium citrate buffer.

Advantageously, the pH is obtained with acetic acid/sodium acetate or citric acid/sodium citrate buffer solutions with molarity of between 0.05 M and 0.2 M.

Even more advantageously, the pH buffer consists of the acetic acid/sodium acetate buffer and the pH of the composition is then 3.5, or the pH buffer consists of the citric acid/sodium citrate buffer and the pH of the composition is then 4.

Advantageously, the composition according to the present invention contains vinflunine ditartrate with a base vinflunine concentration of between 1 and 50 mg/ml, advantageously between 25 and 30 mg/ml and in particular 25 mg/ml or 30 mg/ml. This concentration is thus expressed as base vinflunine. The administered amount depends on the body surface area of the patients.

In one advantageous embodiment, the composition according to the present invention corresponds to one of the following formulations: 68.35 mg of vinflunine ditartrate qs 2 ml in water, or 136.70 mg of vinflunine ditartrate qs 4 ml of water, or 341.75 mg of vinflunine ditartrate qs 10 ml of water, the amount of vinflunine ditartrate corresponding, respectively, in each of the formulations to 50 mg of base vinflunine, 100 mg of base vinflunine and 250 mg of base vinflunine. These data are collated in Table 1 below.

TABLE 1
Examples of unit compositions of the aqueous solution

| Name of the components | Vinflunine unit doses | | | | | |
|---|---|---|---|---|---|---|
| Vinflunine ditartrate | 68.35 | mg | 136.70 | mg | 341.75 | mg |
| corresponding to base | 50.00 | mg | 100.00 | mg | 250.00 | mg |
| vinflunine | | | | | | |
| Water for injection | qs 2 | ml | qs 4 | ml | qs 10 | ml |

Table 1 above shows the possibility of preparing in bottles 3 unit doses of vinflunine resulting from the distribution into different volumes of the same aqueous vinflunine ditartrate solution at a concentration of 25 mg/ml expressed in terms of base vinflunine.

In another embodiment of the invention, the composition according to the present invention remains stable for at least 36 months at 5° C.±3° C.

In one particular embodiment of the invention, the pharmaceutical composition according to the present invention is administered by intravenous infusion, after being dissolved in infusion solutions such as 0.9% sodium chloride or 5% glucose solutions.

The present invention thus also relates to the pharmaceutical composition according to the present invention for its use as a medicinal product, in particular for treating cancer, advantageously for a parenteral administration, advantageously via intravenous infusion, and more advantageously during chemotherapy as an antineoplastic and antitumoral agent.

The present invention also relates to the use of a composition according to the present invention for the manufacture of a medicinal product for parenteral administration, advantageously via intravenous infusion, which is advantageously intended for treating cancer.

The parenteral administration, especially intravenously, of a pharmaceutical vinflunine composition according to the present invention makes it possible to treat cancers that are sensitive to the action of vinflunine.

The present invention also relates to a process for preparing a composition according to the present invention, comprising the following successive steps:
(a) dissolution of the vinflunine salt in water for injection,
(b) optional addition of a pH buffer,
(c) sterilization by filtration of the bulk solution.

In one particular embodiment of the invention, the process according to the present invention comprises the additional step (d) of aseptic distribution, under a nitrogen atmosphere, of the sterile composition obtained in step (c) in a container. Advantageously, this container is chosen from glass phials, preferably of amber or colourless type I, glass bottles, preferably of amber or colourless type I equipped with an elastomeric stopper and a crimped aluminium cap or any compatible ready-to-use system, for instance a prefilled syringe.

The present invention thus also relates to a packaging container containing the composition according to the present invention.

This packaging container may be chosen from glass phials, preferably of amber or colourless type I, glass bottles preferably of amber or colourless type I equipped with an elastomeric stopper and a crimped aluminium cap or any compatible ready-to-use system, for instance a prefilled syringe.

The examples that follow are given as non-limiting indications.

### EXAMPLE 1

### Comparison of the Stability of Vinflunine Ditartrate in Pulverulent Form with that of Vinflunine Ditartrate in Aqueous Solution (Composition According to the Present Invention)

Table 2 below shows the stability results obtained for a batch of pulverulent lyophilized vinflunine ditartrate (batch 503) and a batch of aqueous solution containing 25 mg/ml of base vinflunine (batch SB0222) manufactured with this same batch of vinflunine ditartrate, after 3 months and 6 months of storage at 25° C. The stability is monitored by observing the changes in the total amount of vinflunine-related impurities present.

TABLE 2
vinflunine ditartrate/aqueous solution stability results

| | | Aqueous solution of vinflunine |
|---|---|---|
| | Vinflunine ditartrate | ditartrate (25 mg/ml |
| | (batch 503) | base vinflunine) batch SB0222) |
| | (% impurity relative to | (% impurity relative |
| | 100% of active principle) | to 100% active principle) |
| t₀ | 1.17 | 1.23 |
| t_{3 months} | 2.75 | 1.45 |
| t_{6 months} | 3.48 | 2.00 |

After storage for 6 months at 25° C., the total amount of vinflunine-related impurities increased by:
62% in the aqueous vinflunine ditartrate solution,
197% for the pulverulent vinflunine ditartrate.

### EXAMPLE 2

### Study of Stability as a Function of the pH of the Compositions According to the Present Invention

Stability studies were performed on aqueous vinflunine ditartrate solutions, in a pH range of between 2.5 and 5.0 and more particularly between 3.0 and 4.0. The pH was obtained with 0.2 molar acetic acid/sodium acetate or citric acid/sodium citrate buffer solutions.

The percentage formulations used are presented in Table 3 below. They correspond to a base vinflunine concentration of 30 mg/ml.

TABLE 3
Formulations of buffered aqueous solutions

| | Compositions | | | | | |
|---|---|---|---|---|---|---|
| | BS1332 | | BS1330 | | BS1327 | |
| | (pH = 3.5) | | (pH = 3.5) | | (pH = 4.0) | |
| Vinflunine ditartrate | 4.101 | g | 4.101 | g | 4.101 | g |
| Corresponding to base | 3 | g | 3 | g | 3 | g |
| vinflunine | | | | | | |
| Glacial acetic acid | 1.185 | g | | | | |
| Sodium acetate | 0.100 | g | | | | |
| Citric acid monohydrate | | | 2.885 | g | 2.460 | g |
| Sodium citrate dihydrate | | | 1.903 | g | 2.497 | g |
| Water for injection | qs 100 | ml | qs 100 | ml | qs 100 | ml |

The results were compared with those concerning a simple vinflunine ditartrate aqueous solution, without addition of buffer solution, stored under the same conditions. The pH of this solution is equal to 3.5.

The composition and references of the test solutions are collated in Table 4 below.

TABLE 4
Composition and reference of the test solutions

| | Formulation |
|---|---|
| Composition | reference |
| Solution at pH = 2.5 (citrate buffer) | BS 1325 |
| Solution at pH = 3 (citrate buffer) | BS 1326 |
| Solution at pH = 3.5 (citrate buffer) | BS 1330 |
| Solution at pH = 4 (citrate buffer) | BS 1327 |
| Solution at pH = 5 (citrate buffer) | BS 1328 |
| Solution at pH = 3.5 (citrate buffer) | BS 1332 |
| Unbuffered aqueous solution | BS 1331 |

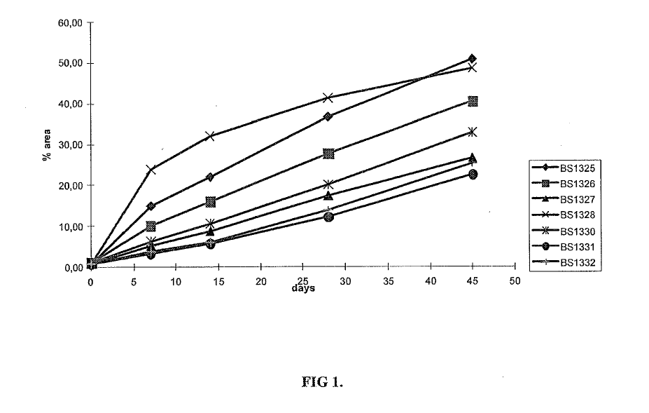

FIG. 1 shows the changes, determined by HPLC, of the content of total vinflunine-related impurities as a function of time, under severe conditions (45 days at 60° C.), for each formulation indicated in Table 3.

They are complemented by the results indicated in Table 5 below, showing the change in colour of the solutions over 7 days at 60° C.

The monitoring of the absorbance of these solutions, in the ultraviolet range, at 410 nm, reveals the appearance of vinflunine oxidation derivatives not chromatographed by HPLC.

TABLE 5
Change in absorbance

Absorbance at 410 nm

| Batch | t₀ | t_{7 days} |
|---|---|---|
| BS 1325 | 0.021 | 0.645 |
| pH = 2.5 | | |
| Citrate buffer: 0.2M | | |
| BS 1326 | 0.020 | 0.520 |
| pH = 3.0 | | |
| Citrate buffer: 0.2M | | |
| BS 1330 | 0.020 | 0.354 |
| pH = 3.5 | | |
| Citrate buffer: 0.2M | | |
| BS 1327 | 0.023 | 0.346 |
| pH = 4.0 | | |
| Citrate buffer: 0.2M | | |
| BS 1328 | 0.020 | 0.896 |
| pH = 5.0 | | |
| Citrate buffer: 0.2M | | |
| BS 1332 | 0.021 | 0.226 |
| pH = 3.5 | | |
| Acetate buffer: 0.2M | | |
| BS 1331 | 0.019 | 0.171 |
| pH = 3.5 | | |
| No buffer | | |

Only the unbuffered solution, pH=3.5, has an absorbance of less than 0.200 after 7 days at 60° C.

The results indicate that the stability of vinflunine is better with a pH value of between 3.0 and 4.0 but is dependent on the nature of the ions of which the buffer is composed. At pH 3.5, the acetic acid/sodium acetate buffer affords better stability than the citric acid/sodium citrate buffer. For the latter buffer, the results are better at pH 4.0.

It is found, entirely surprisingly, that the stability of the aqueous vinflunine ditartrate solution, at its spontaneous pH of 3.5, is better than the stability of vinflunine ditartrate aqueous solutions buffered to pH 3.5.

These good results are confirmed by the long-term stability results collated in Table 6 below, which indicate that the injectable aqueous vinflunine pharmaceutical composition according to the present invention may be stored for at least 36 months at 5° C.±3° C. without undergoing any substantial degradation.

TABLE 6
Stability results of the aqueous pharmaceutical composition
according to the present invention

| | t₀ | t_{3 months} | t_{6 months} | t_{12 months} | t_{24 months} | t_{36 months} |
|---|---|---|---|---|---|---|
| Batch | 30.8 | 30.4 | 30.4 | 30.4 | 30.3 | 30.2 |
| CLP004 | | | | | | |
| Vinflunine | | | | | | |
| ditartrate | | | | | | |
| in | | | | | | |
| water for | | | | | | |
| injection | | | | | | |
| with | | | | | | |
| Vinflunine | | | | | | |
| content in | | | | | | |
| mg/ml | | | | | | |
| (theory = | | | | | | |
| 30.0) | | | | | | |

### EXAMPLE 3

### Stability of Different Aqueous Pharmaceutical Compositions Containing Vinca Alkaloids

Different aqueous pharmaceutical compositions containing vinflunine, vinorelbine, vincristine or vinblastine have been prepared by addition of the alkaloid at a concentration of the base alkaloid of 25 mg/ml to water for injection with or without the use of a buffer system and with or without inerting the composition with N₂.

Their characteristics are as follows:

| | | | Buffer | pH at |
|---|---|---|---|---|
| | Alkaloid | Inerting | system | t = 0 |
| Composition 1 | Vinflunine | no | no | 3.5 |
| according to the | ditartrate | | | |
| present invention | | | | |
| Composition 2 | Vinflunine | yes | no | 3.5 |
| according to the | ditartrate | | | |
| present invention | | | | |
| Comparative | Vinorelbine | no | no | 3.5 |
| composition 3 | ditartrate | | | |
| Comparative | Vinorelbine | yes | no | 3.5 |
| composition 4 | ditartrate | | | |
| Comparative | Vincristine | no | no | 3.5 |
| composition 5 | sulphate | | | |
| Comparative | Vincristine | yes | no | 3.5 |
| composition 6 | sulphate | | | |
| Comparative | Vincristine | no | yes with | 4.0 |
| composition 7 | sulphate | | Acetic | |
| | | | acid- | |
| | | | sodium | |
| | | | acetate | |
| | | | buffer | |
| | | | 0.2M | |
| Comparative | Vincristine | yes | yes with | 4.0 |
| composition 8 | sulphate | | acetic | |
| | | | acid- | |
| | | | sodium | |
| | | | acetate | |
| | | | buffer | |
| | | | 0.2M | |
| Comparative | Vinblastine | no | yes with | 3.6 |
| composition 9 | sulphate | | acetic | |
| | | | acid- | |
| | | | sodium | |
| | | | acetate | |
| | | | buffer | |
| | | | 0.2M | |
| Comparative | Vinblastine | yes | yes with | 3.6 |
| composition 10 | sulphate | | acetic | |
| | | | acid- | |
| | | | sodium | |
| | | | acetate | |
| | | | buffer | |
| | | | 0.2M | |
| Comparative | Vinblastine | no | yes with | 4.0 |
| composition 11 | sulphate | | acetic | |
| | | | acid- | |
| | | | sodium | |
| | | | acetate | |
| | | | buffer | |
| | | | 0.2M | |
| Comparative | Vinblastine | yes | yes with | 4.0 |
| composition 12 | sulphate | | acetic | |
| | | | acid- | |
| | | | sodium | |
| | | | acetate | |
| | | | buffer | |
| | | | 0.2M | |

After one month of storage of these compositions under the following conditions +2/+8° C. or +40° C. and 75% of relative humidity, the following tests were performed:

| Test | Method |
|---|---|
| Appearance of the solution | Visual observation |
| Determination of pH | European Pharmacopoeia 2.2.3 (on |
| | the solution as is) |
| Degradants by UV: absorbance | European Pharmacopoeia 2.2.25 |
| at 420 nm | (on the solution as is) |
| Impurities/degradants for | Liquid chromatography using a |
| vinflunine formulae | reverse phase column and a |
| | gradient of solvents |
| Impurities/degradants for | Liquid chromatography based on |
| vinorelbine formulae | European Pharmacopoeia 2107 |
| | monograph |
| Impurities/degradants for | Liquid chromatography based on |
| vincristine formulae | vinblastine sulphate European |
| | Pharmacopoeia 0748 monograph |
| Impurities/degradants for | Liquid chromatography based on |
| vinblastine formulae | vinblastine sulfate European |
| | Pharmacopoeia |

The results are as follows:

pH

| INERTING | YES | | NO | |
|---|---|---|---|---|
| STORAGE | +2°/+8° C. | +40° C. | +2°/+8° C. | +40° C. |
| CONDITIONS | | 75% RH | | 75% RH |
| (1 MONTH) | | | | |
| VINFLUNINE | Composition 2 | | composition 1 | |
| DITARTRATE | 3.4 | 3.4 | 3.4 | 3.4 |
| VINORELBINE | comparative | | comparative | |
| DITARTRATE | composition 4 | | composition 3 | |
| | 3.5 | 3.4 | 3.5 | 3.5 |
| VINCRISTINE | comparative | | comparative | |
| SULPHATE | composition 6 | | composition 5 | |
| | 3.5 | 2.8 | 3.3 | 2.7 |
| | comparative | | comparative | |
| | composition 8 | | composition 7 | |
| | (buffer pH 4.0) | | (buffer pH 4.0) | |
| | 3.9 | 3.9 | 3.9 | 3.8 |
| VINBLASTINE | comparative | | Comparative | |
| SULPHATE | composition 10 | | composition 9 | |
| | (buffer pH 3.6) | | (buffer pH 3.6) | |
| | 3.6 | 3.5 | 3.5 | 3.5 |
| | comparative | | comparative | |
| | composition 12 | | composition 11 | |
| | (buffer pH 4.0) | | (buffer pH 4.0) | |
| | 3.9 | 3.9 | 3.9 | 3.9 |

The pH remains stable over the test period at either +2°/+8° C. or +40° C. 75% RH whether inerted or not for the solutions of vinflunine, vinblastine and vinorelbine.

There is a decrease of the pH value for the two unbuffered vincristine formulae.

APPEARANCE OF THE SOLUTION

| INERTING | YES | | NO | |
|---|---|---|---|---|
| STORAGE | +2°/+8° C. | +40° C. | +2°/+8° C. | +40° C. |
| CONDITIONS | | 75% RH | | 75% RH |
| (1 MONTH) | | | | |
| VINFLUNINE | Composition 2 | | Composition 1 | |
| DITARTRATE | Colourless | Pale | Colourless | Pale |
| | solution | yellow | solution | yellow |
| | | solution | | solution |
| VINORELBINE | Comparative | | Comparative | |
| DITARTRATE | composition 4 | | composition 3 | |
| | Colourless | Yellow | Colourless | Yellow |
| | solution | solution | solution | solution |
| VINCRISTINE | Comparative | | Comparative | |
| SULPHATE | composition 6; | | composition 5; | |
| | comparative | | comparative | |
| | composition 8 | | composition 7 | |
| | (buffer pH 4.0) | | (buffer pH 4.0) | |
| | Colourless | Yellow to | Colourless | Yellow to |
| | solution | orange | solution | orange |
| | | solution | | solution |
| VINBLASTINE | Comparative | | Comparative | |
| SULPHATE | composition 10 | | composition 9 | |
| | (buffer pH 3.6); | | (buffer pH 3.6); | |
| | comparative | | comparative | |
| | composition 12 | | composition 11 | |
| | (buffer pH 4.0) | | (buffer pH 4.0) | |
| | Colourless | Yellow | Colourless | Yellow |
| | solution | solution | solution | solution |

Compared to the vinflunine solution, the solutions of vinorelbine, vinblastine and vincristine showed a significant to intense yellowing when stored for one month at 40° C. and 75% RH.

ABSORBANCE A 420 nm

| INERTING | YES | | NO | |
|---|---|---|---|---|
| STORAGE | +2°/+8° C. | +40° C. | +2°/+8° C. | +40° C. |
| CONDITIONS | | 75% RH | | 75% RH |
| (1 MONTH) | | | | |
| VINFLUNINE | Composition 2 | | Composition 1 | |
| DITARTRATE | 0.014 | 0.051 | 0.014 | 0.072 |
| VINORELBINE | Comparative | | Comparative | |
| DITARTRATE | composition 4 | | composition 3 | |
| | 0.042 | 0.136 | 0.040 | 0.227 |
| VINCRISTINE | Comparative | | Comparative | |
| SULPHATE | composition 6 | | composition 5 | |
| | 0.022 | 0.293 | 0.022 | 0.486 |
| | Comparative | | Comparative | |
| | composition 8 | | composition 7 | |
| | (buffer pH 4.0) | | (buffer pH 4.0) | |
| | 0.032 | 0.550 | 0.035 | 0.650 |
| VINBLASTINE | Comparative | | Comparative | |
| SULPHATE | composition 10 | | composition 9 | |
| | (buffer pH 3.6) | | (buffer pH 3.6) | |
| | 0.032 | 0.194 | 0.038 | 0.315 |
| | Comparative | | Comparative | |
| | composition 12 | | composition 11 | |
| | (buffer pH 4.0) | | (buffer pH 4.0) | |
| | 0.038 | 0.315 | 0.043 | 0.389 |

Compared to the vinflunine solution, the increase in absorbance is: ×3 with the vinorelbine solution, ×7 to 12 with the vincristine solution and ×4 to 6 with the vinblastine solution.

In conclusion, the absorbance of the solution, related to the observed color, is not acceptable for an aqueous injectable formulation at 25 mg of base/ml for vinorelbine, vinblastine and vincristine.

In particular, vinflunine ditartrate is more stable in an aqueous solution which does not contain any buffer pH or any preservatives at 25 mg/ml than vinorelbine.

This is quite surprising since the solubility of vinorelbine is higher than the solubility of vinflunine (respectively 1000 mg/ml and between 290 and 330 mg/ml) and therefore the stability of vinorelbine in water should have been higher at a high concentration (25 mg/ml) than the one of vinflunine.

Considering the required vinflunine concentration of 25 mg of base/ml, the knowledge on the aqueous stability of other vinca alkaloids could not predict a vinflunine ditartrate formulation in water as being the better choice at said concentration for an injectable formulation.

1. Method for treating cancer comprising the parenteral administration of an effective amount to a patient in need thereof of a composition of vinflunine, wherein said composition is in the form of a sterile injectable aqueous solution of vinflunine ditartrate without the addition of a buffer system.

2. Method according to claim 1 wherein the composition contains vinflunine ditartrate with a base vinflunine concentration of between 25 and 30 mg/ml.

3. Method according to claim 2 wherein the concentration of vinflunine base is of 25 mg/ml.

4. Method according to claim 1 wherein the pH of said composition is between 3.0 and 4.0.

5. Method according to claim 3 wherein the pH of said composition is 3.5.

6. Method according to claim 1 wherein the composition has been prepared up to 36 months before its administration.

7. Method according to claim 1 or 6 wherein the composition has been stored at 5° C.±3° C.

8. Method according to claim 1 wherein the parenteral administration is via intravenous infusion.

9. Method according to claim 1 wherein the composition corresponds to one of the following formulations:
68.35 mg of vinflunine ditartrate qs to 2 ml in water, or
136.70 mg of vinflunine ditartrate qs to 4 ml of water or
341.75 mg of vinflunine ditartrate qs to 10 ml of water

10. Method according to claim 1 wherein the composition has been prepared in the following way:
(a) dissolution of vinflunine ditartrate in water for injectable preparations,
(b) sterilization by filtration of the bulk solution,
(c) aseptic distribution, under a nitrogen atmosphere, of the sterile composition obtained in step (c) in the container, advantageously chosen from glass phials, glass bottles and prefilled syringes.

11. Method according to claim 1 wherein the composition is contained in a packaging container.
